(19) United States
(12) Patent Application Publication
(10) Pub. No.: US 2014/0113356 A1
(43) Pub. Date: Apr. 24, 2014

(54) NANO-ELECTRODE BASED CHIP

(71) Applicant: NATIONAL TSING HUA UNIVERSITY, HSINCHU (TW)

(72) Inventors: Fan-Gang TSENG, New Taipei City (TW); Tuhin Subhra SANTRA, Hsinchu (TW)

(73) Assignee: NATIONAL TSING HUA UNIVERSITY, HSINCHU (TW)

(21) Appl. No.: 13/753,036

(22) Filed: Jan. 29, 2013

(30) Foreign Application Priority Data
Oct. 23, 2012 (TW) 101139140

(51) Int. Cl. C12N 13/00

(52) U.S. Cl. CPC C12N 13/00; Y10S 977/906; Y10S 977/811; B82Y 5/00
USPC 435/283.1; 977/906; 977/811

(57) ABSTRACT

The present invention disclosed a nano-electrode based transparent chip, which is applicable to perform a micro fluidic substance diffused through a specific region of a single cell membrane by localized electroporation technique with membrane reversibility. The chip comprises a silicon-based layer, different structural layers, an insulating layer and a micro fluidic layer. When the individual single cell was placed on the gap between a plurality of triangular-shaped nano-electrodes with nano-tip, then electric field can intense on a specific region of the individual single cell causes the formation of nano-pores on the membrane, resulting to deliver drugs, DNA molecules from outside of the cell to the inside of the cell with a high transfection rate and a high cell viability. This technique not only generates well-controlled nano-pores to allow rapid recovery of the cell membrane, but also provides clear optical path for potentially monitoring/tracing the drugs into the cell.

### CROSS-REFERENCE TO RELATED APPLICATION

This application claims the benefit under 35 U.S.C. §119 of Taiwanese Patent Application No. 101139140, filed Oct. 23, 2012, which is hereby incorporated by reference in its entirety.

### BACKGROUND

1. Field of the Invention

The present invention relates to the field of a nano-electrode based transparent chip, in particular to apply an intense electric field for localized electroporation toward the individual single cell by using nano-gap with nano-electrodes, which are very smaller than the size of the single cell to be tested for achieving the goal, to deliver drugs/medicine through a nano region of single cell without destroying the single cell membrane structure.

2. Description of the Related Art

The delivery of drugs into cell is an important phenomenon for biological studies and therapeutic applications. The gene delivery technique which is related to it includes a plurality of different methods and means. Although viral transfection being one of the traditional method which is widely acceptable and effective method for drug delivery, but it may cause some problems such as response of immunity, low controlling rate and so on. These reasons resulting, this technique has some chances to be improved.

In addition, the methods of the non-viral transfection used for gene delivery technique including, jet injection, lipid mediated entry into cells, sonoporation, or electroporation. The conventional electroporation may cause some problems such as fusing or collapsing of the cell membrane structure easily, so that genes or drugs could not be effectively and fast, delivered inside of the single cell. Furthermore, the conventional method is difficult to be controlled in the polar direction. Thus, it is an ineffective to control the injecting position of genes or drugs. Due to affected of whole cell membrane by electric field, it is also impossible to control the doses of drugs delivery inside the cell by using conventional electroporation technique.

Therefore, a precisely drug delivery systems of the non-viral transfection through a specific region of the single cell is a main topic in the field of single cell research and development in recent years. Wherein it includes the electroporation by utilizing a high intense electric field. Many conventional techniques generate an AC/DC electric field by using two large electrodes to permeabilize the cell membrane structure cause to form membrane nano-pores. However, these methods usually cause some problems such as large electrodes area can generate more hydroxyl and hydrogen ion causes more toxicity of the cell environment resulting to reduce the cell viability. Also these methods have some problem for the cell membrane resealing, for example, its reversibility being disappeared and its structure being collapsed, finally cell death.

Therefore, it is urgent and necessary to provide a method to apply an intense electric field through a specific region (submicron to nano region) of single cell membrane to deliver drugs inside the individual single cell to achieve the goal of upgrading the transmittance of medicine without destroying the whole membrane structure of the single cell.

### BRIEF SUMMARY

Based on the aforementioned problems in the prior art technique, one objective of the present invention is to provide a nano-electrode based transparent chip with nano-gap between two electrodes to overcome the defect of inaccurate delivery of drugs into the single cell. This nano-gap with nano-electrodes resulting fully controllable drug delivery through a specific region of single cell with membrane reversibility to deliver specific doses of drugs into the single cell and avoided to collapse of the cell membrane structure with negligible cell toxicity, when the cell is electroporated.

The present invention comprises a silicon-based layer, a structural layer, an insulating layer, and a micro fluidic layer. The structural layer is deposited on the silicon-based layer, and a plurality of nano-electrodes is deposited on the structural layer. The insulating layer is deposited on the top of the plurality of nano-electrodes. Then final nano-gap with nano-hole is form, in between two nano-electrodes throughout the insulating and structural layer. This nano-gap is form in all of plurality of nano-electrodes. As nano-gap form throughout the insulation and structural layer which is nothing but nano-hole form in between two nano-electrodes. When the individual single cell is disposed on the nano-gap between the plurality of nano-electrodes, which is able to generate at least an intense electric field on specific region of single cell membrane, then localized single cell membrane can deform to permeabilize the drugs or medicine through the nano-hole in each plurality of nano-gap electrodes. The drugs diffuse through the nano-hole by microfluidic nano-channel and it is enter into the localized single cell through the specific region of membrane nano-pores.

The size of the nano-gap between the plurality of nano-electrodes is in the range from 25 nanometers to 500 nanometers. As the 25 nanometer gap enhance much more an intense electric field to compare with 500 nm nano-gap between plurality of nano-electrodes. To enhance much more intense electric field on local area (25 nm), which affect the localized cell membrane area is less with compare 500 nm nano-gap between the plurality of nano-electrodes. As results the drugs or medicine can enter with more controllable doses for 25 nanometer gap between the plurality of nano-electrodes.

Preferably, the micro fluid flows within the plurality of nano-holes through the nano-channel and the nano-gap between two nano-electrodes.

Preferably, the plurality of nano-electrodes are respectively applied with a single positive square wave pulse to generate at least an intense electric field.

Preferably, the plurality of nano-electrodes is indium tin oxide (ITO) nano-electrodes.

Preferably, the plurality of nano-electrodes is nano-electrodes with triangular shape nano-tips, which form an intense electric field at specific point.

Preferably, the insulating layer is a silicon oxide (SiO₂) layer to inhibit hydroxyl and hydrogen ion generation which can avoid toxic issue.

Preferably, the micro fluid circulates through at least one of the plurality of nano-hole with nano-channel through a plurality of nano-gap with nano-electrodes.

Preferably the structural layer, insulating layer and a transparent layer are further deposited on a portion of a bottom of the silicon-based layer.

Preferably, the silicon-based layer, the structural layer, insulating layer and the transparent layer define a micro fluidic chamber, where to allow the micro fluid flow in and out of the micro fluidic chamber through the microfluidic channel.

In summarization of the description above, the nano-electrode based transparent chip of the present invention includes one or more advantages as follows:

(1) The nano-electrode based transparent chip of the present invention is applicable to improve the localize drugs delivery into the single cell without destroying much more area of the single cell membrane structure which enhance the transfection efficiency and viability of the single cell.

(2) The nano-gap with nano-electrodes reduces the hydrogen and hydroxyl ion generation during electroporation experiment. Moreover the insulation layer protect the ion generation from the nano-electrodes resulting to reduces the toxicity effect on the cell membrane and enhance the cell viability.

(3) Through controlling the strength of the electric field, electroporation gets a better control to form the nano-pores on the membrane. The electric field affects such a small region of the single cell membrane, which successfully generate a reversible electroporation with fully controllable drug delivery inside the single cell and transparent chip provides a clear optical path to trace the drugs or DNAs deliver into the cell.

(4) To control electric pulse length, number of pulses and time between two pulses, as results the number of nano-pores opening, the nano-pores size and the density of the nano-pores should be well controlle for localized single cell electroporation.

### BRIEF DESCRIPTION OF THE DRAWINGS

FIG. 1 is a first schematic diagram of a preferred embodiment of the nano-electrode based transparent chip of the present invention.

FIG. 2 is a schematic diagram of the arrangement of electrodes of the nano-electrode based transparent chip of the present invention.

FIG. 3 is a second schematic diagram of a preferred embodiment of the nano-electrode based transparent chip of the present invention.

FIG. 4 is a schematic diagram of the diffusive result of the micro fluid of the nano-electrode based transparent chip of the present invention.

### DETAILED DESCRIPTION

The technical contents and characteristics of the present invention will be apparent with the detailed description of a preferred embodiment accompanied with related drawings as follows. For simplicity, the same numerals are used for the same respective elements in the description of the following preferred embodiments and the illustration of the drawings.

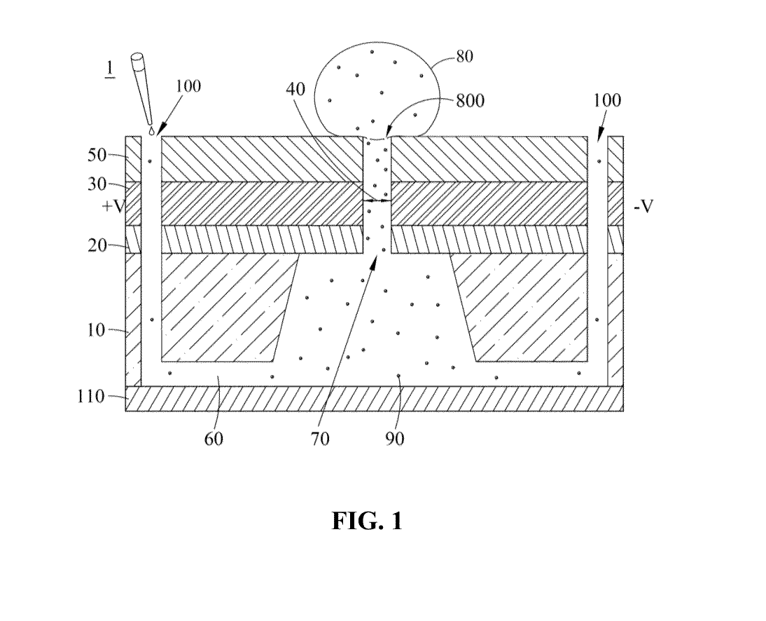

With reference to FIG. 1 for a first schematic diagram of a preferred embodiment of the nano-electrode based transparent chip of the present invention, the nano-electrode based transparent chip 1 comprises a silicon-based layer 10, a structural layer 20, an insulating layer 50, and a micro fluidic layer 60. The structural layer 20 is deposited on the silicon-based layer 10, and a plurality of nano-electrodes 30 are deposited on the structural layer 20. Each nano-hole 40 with nano-channel is formed on every plurality of nano-gap electrodes which penetrating the structural layer 20, plurality of nano-electrode layer 30 and an insulating layer 50. The nano-holes with nano-channel are formed for total nano-gap electrodes 30. The structural layer 20 is a low stress silicon nitride (Si₃N₄) layer or other kinds of layer composed of materials with similar characteristic, but the present invention is not limited to such arrangement only. The plurality of nano-electrodes 30 are indium tin oxide (ITO) material based nano-electrodes or other kinds of nano-electrode composed of materials with similar characteristic, but the present invention is not limited to such arrangement only. The size of the nano-gap 40 between the plurality of nano-electrodes 30 is in the range from 25 nanometers to 500 nanometers. Wherein the insulating layer 50 is deposited on the plurality of nano-electrodes 30. The insulating layer 50 deposited on the plurality of nano-electrodes 30 is a silicon di-oxide (SiO₂) layer or other kinds of layer composed of materials with similar characteristic, but the present invention is not limited to such arrangement only. A plurality of microfluidic channel 100 is form with micro fluidic layer 60 and a portion 70 of the plurality of microfluidic nano-channel is in communication with the nano-hole 40 with nano-channel. That is, like two microfluidic channel 100 are disposed in the present embodiment, the fresh micro fluid 90 can be injected into one of them through the microfluidic channel 100, and the used micro fluid 90 can be released out from the other microfluidic channel 100 after the experiment. That makes the circulation of micro fluid 90 effective much more. On the other hand, the micro fluid 90 could keep fresh by using the communication between at least one portion 70 and the nano-hole 40 with nano-channel to circulate and the micro fluid 90 could be used much more effectively.

In addition, in the present embodiment, when the individual single cell 80 is disposed on the nano-gap 30 in between the plurality of nano-electrodes 30 which is able to generate at least an intense electric field, then the electric field acts on the individual single cell membrane 80 to cause the localize electroporation on a specific position of cell membrane of the individual single cell 80 results to form nano-pores 800. Therefore, a portion of the micro fluid 90 flows from the plurality of the microfluidic channel 100 and passing through the nano-hole with nano-channel 40 between the plurality of nano-gap electrodes 30 and then later flows into the individual single cell 80 through the localize area 800 after passing through the portion of nano-channel 70. More specifically, when the micro fluid flows through the nano-channel 40 disposed between the plurality of nano-electrodes 30 from the portion 70 and then passes through a plurality of nano-pores on the cell membrane 800 of the individual single cell 80, one or the combination of a biomolecular material with nano size, a molecular drug and a molecular probe contained in the micro fluid 90 could be delivered into the individual single cell 80 for further observation.

It is noteworthy that because the size of the nano-hole with nano-channel 40 disposed between the plurality of nano-gap electrodes 30 is smaller than that of the individual single cell 80, the electric field intense by the nano-gap electrodes 30 works merely a range such as it acts on a small portion of the cell membrane of the individual cell 80 to cause the localize single cell electroporation. Therefore, a plurality of nano-pores 800 generated on the single cell membrane by using nano-electrode based transparent chip of the present invention for successful reversible electroporation. That is, those nano-pores would not produce any effect on the main structure of the whole body of the individual single cell 80. More specifically, when the micro fluid 90 passes though the plurality of nano-pores 800 on the cell membrane of the individual single cell 80 with electroporation technique, after some time of successful localize electroporation, the individual single cell 80 where the plurality of nano-pores 800 on the cell membrane are disappeared and the cell membrane recovers back to its original condition. Therefore, this is called a reversible electroporation. After reversible electroporation, the intensity inside the single cell 80 should be constant, because no more drugs can enter inside the single cell 80.

In addition, the electric field intense by applying the positive voltage (+V) and negative voltage (−V) on the plurality of nano-electrodes can be in the form of electric pulse. Preferably, it can be a square wave electric pulse with much more preferably, it can be a single positive square wave electric pulse. The user can control pulse length, number of pulses, time between two pulses, as results the number of nano-pores opening, the nano-pores size and the density of the nano-pores should be controlled on nanopores 800 of the individual single cell 80 for corresponding to the size of the molecule, the drug, the probe or the biomedical material included in the micro fluid 90. The user wants to deliver drugs into an individual single cell 80 through adjusting the frequency, the field strength and the duration of the electric field. Furthermore, in the nano-hole with nano-channel 40, the pulse of electric field generated by the plurality of nano-electrodes 30 with nano-gap (25 nm) is highly intense, and the effective area of the pulse of electric field is minimized because of nano-gap between two nano-electrodes 30 which extremely lowering the damage toward the individual single cell 80.

In addition, the bottom layer of the micro fluidic layer 60, structural layer 20, nano-electrodes layer 30 and oxide layer 50 of the nano-electrode based transparent chip 1 of the present invention comprises a fully light transparent layer. The advantage of adopting the light transparent layer 110 is that, when the molecular drug or material included in the micro fluid 90 flows from the micro fluidic layer 60 to moves toward the individual single cell 80, the sensor connected from the outside, senses and receives variable signals, particularly the optical signals, through the light transparent layer 110 when the nano-electrode based transparent chip 1 is in operation mode. For example, if the micro fluid 90 comprises the drug emission fluorescence like propidium iodide (PI)/calcein/quantum dot (QD) or GFP flow into the individual single cell 80, the biomolecules insert into the individual single cell 80 and then gradually accumulate inside the individual single cell 80. When the plurality of nano-pores 800 on the cell membrane of the individual single cell 80 is resealed and the cell membrane recovers back to its original condition, then biomolecules are left inside the individual single cell 80. At the same time, the sensor connected from outer place immediately detects the variation of the fluorescent signals coming from the biomolecules when it flows like the process mentioned above and then obtains the usage effect of the nano-electrode based transparent chip 1 of the present invention Also the variation of fluorescence intensity of the single cell can confirm the resealing mode of nano-pores 800, where the invertor used Inverted fluorescence microscope for which the chip with all layers should be needed to be transparent. This nano-electrodes based transparent chip should be comfortable for electroporation experiment in both inverted and non-inverted fluorescence microscope.

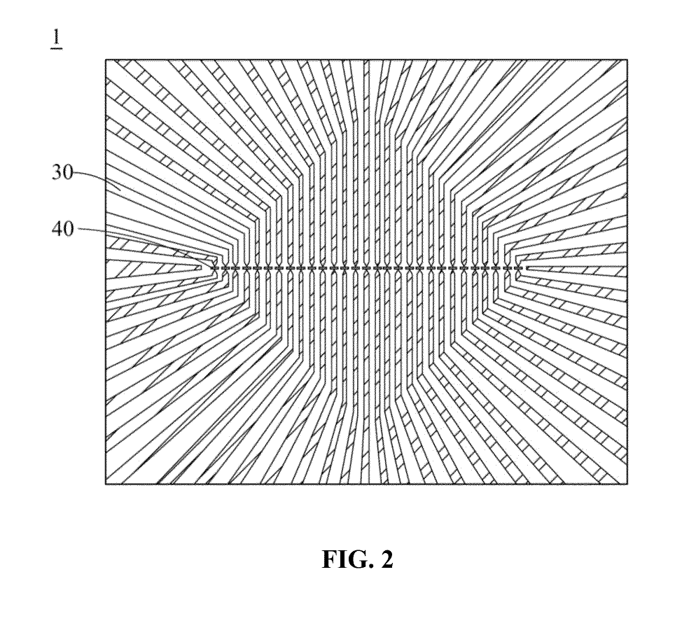

With reference to FIG. 2 for a schematic diagram of the arrangement of the nano-electrode based transparent chip 1 in the present invention. The plurality of nano-gap electrodes 30 included in the nano-electrode based transparent chip 1 of the present invention are designed with the triangular shape nano-tips and arranged in the form of radiation, concentric circle or matrix, etc. In order to describe easily, the present embodiment adopts the design of triangular shape nano-tip with nano-gap 30 between two nano-electrodes can enhance much more an intense electric field into the nano region of the single cell membrane with comparison of any rectangular or other shape of electrodes. But the present invention is not limited to such arrangement only. It is noteworthy that the nano-hole with nano-channel 40 is preferably generated by the way of focus ion beam (FIB). The nano-hole with nano-channel 40 is disposed on the geometric center of the intervals between the plurality of nano-gap electrodes 30 to achieve better electroporation efficiency resulting from the intense electric field toward the individual single cell 80. Additionally, when the distance of the nano-gap 30 between the plurality of nano-electrodes becomes smaller, the generated electric field is more intense. The strength is further changed in accordance with the external voltage amplitude acted on the plurality of nano-electrodes 30 to correspond to the different characteristics of the cell membrane of the individual single cell 80 needed to be electroporated. That is, the user could adjust the parameters suitable to different telecommunication and the distance of the nano-gap electrodes toward different samples of an individual single cell 80 needed to be tested by using the nano-electrode based transparent chip 1 of the present invention, then achieve the best result.

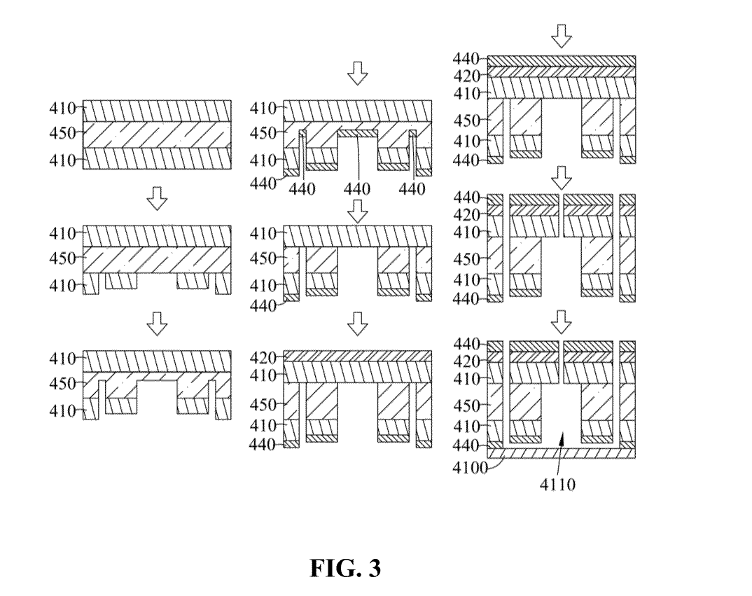

With reference to FIG. 3 for a schematic diagram of a preferred embodiment of the nano-electrode based transparent chip of the present invention, the top side and the bottom side of the silicon based layer 450 are respectively deposited a structural layer 410. Then the structural layer 410 on the bottom side of the silicon based layer 450 is etched to generate a predetermined pattern which is changed according to the requirement of the user. For example, if the user needs a plurality of microfluidic channel on the silicon based layer 450, a plurality of predetermined patterns are etched. Then another insulating layer 440 is deposited on structural layer 410 on the bottom side of the silicon based layer 450. After deposit layer 440, the unwanted silicon layer on 450 is etched from the bottom side until the top layer 410 appeared. Then transparent Indium Tin Oxide (ITO) layer 420 is deposited on top side of the structural layer 410 and 420 layer is patterned to make as ITO lines. Finally another oxide layer 440 deposited on 420 layer on top side of the chip and then at least one nano-channel 70 and at least one nano-gap with nano-channel 40 are generated by the method of focus ion beam. Then bottom side 440 layer is covered by glass 4100 layer. The final chip is packaged with Printed Circuit Board (PCB). It is noteworthy that the silicon based layer 450, the structural layer 410, another structural layer 410, another insulating layer 440 and the light transparent layer 4100 can define the micro fluidic chamber 4110 comprising at least one nano-channel 70 and microfluidic channel 100 to allow the micro fluid 90 flowing in and out of the micro fluidic chamber 4110 from the microfluidic channel 100.

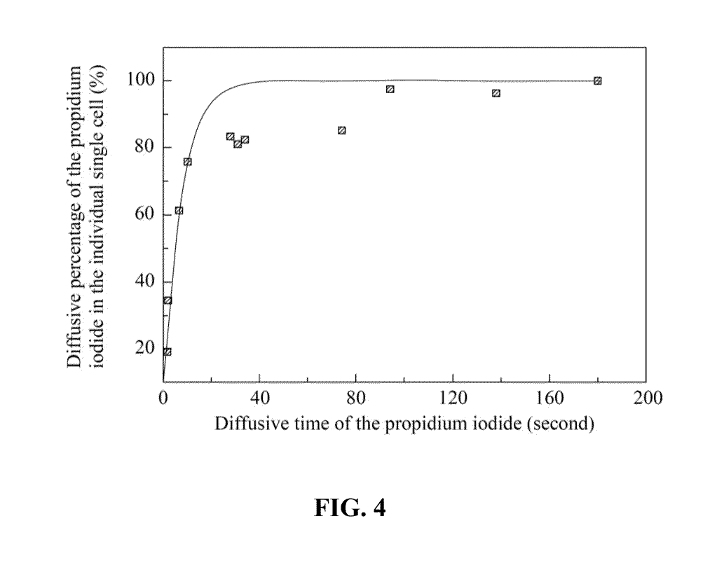

With reference to FIG. 4 for a schematic diagram of the diffusive result of the micro fluid of the nano-electrode based transparent chip of the present invention, the horizontal axis is the diffusive time of the propidium iodide and the vertical axis is the diffusive percentage of the propidium iodide of the individual single cell. In the present embodiment, the micro fluid 90 uses the propidium iodide as the traceable element in practice, but the present invention is not limited to such arrangement only. When the sample of the cell is loaded on the nano-electrode based transparent chip 1 and after the action of localize electroporation, the added propidium iodide in the micro fluidic layer 60 can be over 90 percentage of diffusive rate after around 20 seconds. That is, it ensures the micro fluid 90 can precisely enter into the individual single cell 80 and the individual single cell 80 can keep its completeness at the same time though the nano-electrode based transparent chip 1 of the present invention. Obviously, this technique effectively solves the problems of cell death, avoiding high toxicity, enhance the cell viability and so on resulting from the disappearance of the reversibility of the cell membrane with opening and the lysis in structure of the main body of the cell in the prior art.

What is claimed is:

1. A nano-electrode, with nano-gap, based chip, applicable to an individual single cell to cause a localize electroporation thereon and allow a micro fluid to enter into the individual single cell, the nano-electrode based chip being transparent comprising:
a silicon-based layer;
a structural layer, the structural layer being deposited on the silicon-based layer, with a plurality of nano-electrodes being deposited on the structural layer, and the at least one nano-gap formed on the structural layer disposed between the plurality of nano-electrodes;
an insulating layer, the insulating layer being deposited on the plurality of nano-electrodes, and the insulating layer containing at least one nano-hole with nano-channel penetrating the structural layer and the insulating layer; and
a micro fluidic layer, a plurality of nano-hole being disposed thereon and a portion of the plurality of nano-hole with nano-channel being in communication with the nano-gap,
wherein the individual single cell is disposed on the nano-gap between the plurality of nano-electrodes, which is able to generate at least an intense electric field, then the electric field acts on the individual single cell to cause the localize electroporation on a cell membrane of the individual single cell resulting the formation of plurality of nano-pores thereon, a portion of the micro fluid flows through the nano-channel with nano-hole into the plurality of the nano-pores passing through the nano-gap between the plurality of nano-electrodes earlier and then later flows into the individual single cell after passing through the nano-pores.

2. The nano-electrode based chip of claim 1, wherein the micro fluid flows within the plurality of nano-holes with nano-channel and the nano-gap between two nano-electrodes.

3. The nano-electrode based chip of claim 1, wherein the plurality of nano-electrodes are respectively added a positive voltage and a negative voltage to generate the at least an intense electric field.

4. The nano-electrode based chip of claim 1, wherein the plurality of nano-electrodes are indium tin oxide (ITO) based nano-electrodes.

5. The nano-electrode based chip of claim 1, wherein the plurality of nano-electrodes are nano-electrodes with triangular shape nano-tip which can enhance the intense electric field.

6. The nano-electrode based chip of claim 1, wherein the insulating layer is a silicon dioxide layer (SiO₂).

7. The nano-electrode based chip of claim 1, wherein the micro fluid circulates through the at least one of the plurality of nano-hole with nano-channel and the at least one nano-gap with nano-electrodes.

8. The nano-electrode based chip of claim 1, wherein another one of the structural layers, other insulating layers and a transparent layer are further deposited on a portion of a bottom side of the silicon-based layer.

9. The nano-electrode based chip of claim 1, wherein the silicon-based layer, structural layer, insulating layer and the transparent layer define a micro fluidic chamber comprising at least one microfluidic channel to make the micro fluid flow in and out of the micro fluidic chamber from the plurality of nano-hole.
